(19) United States
(12) Patent Application Publication
(10) Pub. No.: US 2018/0126047 A1
(43) Pub. Date: May 10, 2018

(54) POLYMERIC IMPLANTABLE MEDICAL DEVICES AND SURGICAL INSTRUMENTS

(71) Applicant: SABIC Global Technologies B.V., Bergen op Zoom (NL)

(72) Inventors: Lynn COLUCCI-MIZENKO, Niskayuna, NY (US); Andrew KUGLER, Albany, NY (US)

(21) Appl. No.: 15/574,507

(22) PCT Filed: May 19, 2016
(86) PCT No.: PCT/US16/33325

§ 371 Date: Nov. 16, 2017

Related U.S. Application Data
(60) Provisional application No. 62/163,963, filed on May 19, 2015.

(51) Int. Cl. A61L 31/06; A61L 27/08; A61L 27/10; A61L 27/04; A61L 27/18; A61L 31/02; A61L 31/16; A61L 27/54; A61L 27/28; A61L 31/08; A61B 17/064; A61B 17/16; A61F 2/32; A61F 2/38; A61F 2/44; A61N 1/05; A61F 2/00

(52) U.S. Cl. CPC A61L 31/06; A61F 2002/2835; A61L 27/10; A61L 27/04; A61L 27/18; A61L 31/028; A61L 31/024; A61L 31/022; A61L 31/16; A61L 27/54; A61L 27/28; A61L 31/08; A61B 17/064; A61B 17/1671; A61F 2/32; A61F 2/38; A61F 2/4405; A61N 1/0534; A61F 2/0063; A61L 2300/404; A61L 2300/406; A61L 2300/408; A61L 2430/24; A61L 2430/38; A61L 2430/02; A61F 2002/444; A61L 27/08

(57) ABSTRACT

Devices prepared from polyetherimide resins are disclosed. In one aspect, the article can be medical device configured for use in a body or relating to a medical operation.

### CROSS REFERENCE TO RELATED APPLICATION

This application claims the benefit of U.S. Provisional Application No. 62/163,963, filed May 19, 2015, the entirety of which is incorporated by reference herein.

### TECHNICAL FIELD

The disclosure generally relates to implantable medical devices and surgical instruments having improved properties, and more particularly to implantable medical devices and surgical instruments having improved mechanical strength and biocompatibility.

### BACKGROUND

Implantable medical devices are implanted into the body for various reasons, including hip replacement, spinal procedures, knee replacement, bone fracture repair, etc. Surgical tools and instruments are commonly used in many surgical procedures and are often used to implant medical devices within the body.

In view of the structural integrity requirements of these implantable medical devices and surgical tools, the materials of fabrication are limited, and conventionally include various metal, plastic and composites. Implantable medical devices are usually composed of metals, such as titanium or cobalt chrome alloys, or from polyetheretherketone (PEEK), a polymer that is commonly used in implantable medical devices. These implant materials, however, do not possess sufficient mechanical strength and biocompatibility for all medical devices.

Another problem associated with implantable medical devices is infection, which may in some cases lead to sepsis and death. As a result, it is critical that implantable medical devices and the surgical instruments used to implant them are properly sterilized prior to implantation. Therefore, the devices as well as the surgical instruments must be composed of materials that are not only capable of sterilization prior to surgery, but also highly resistant to infection once they are implanted. Implantable-grade or medical-grade polymeric devices, however, are sensitive to temperature, radiation, and moisture of traditional sterilization processes.

Therefore, there is a need for an implantable medical devices that have biocompatibility, strength, flexibility, wear resistance, and radiolucency yet do not undergo meaningful loss of structural integrity, are not discolored, and do not lose electrical properties as a result of multiple sterilizations.

There is also a need for a polymeric implantable medical device that is capable of being sterilized by radiation, such as gamma and E-beam sterilization procedures. Gamma and E-beam sterilization typically subjects devices to irradiation sterilization but traditional polymeric devices, in particular, will inevitably be affected by the radiation and will experience changes in their polymer structure (such as chain scission and cross-linking). These processes may lead to significant changes and compromise in the tensile strength, elongation at break, and yield strain of such polymeric devices. Furthermore, the exact changes in mechanical properties may not be immediately apparent as there can be some time delay in the development of these changes.

There is a further need for a polymeric implantable medical device that is MRI (magnetic resonance imaging) compatible.

Accordingly, the present disclosure provides such implantable medical devices and surgical instruments that have improved properties over currently existing implantable medical devices and surgical instruments.

### SUMMARY

In accordance with one aspect of the disclosure, an implantable medical device formed from a polymer composition comprising a polyetherimide is disclosed.

In accordance with another aspect of the disclosure, a surgical instrument formed from a polymer composition comprising a polyetherimide is disclosed.

### DETAILED DESCRIPTION

Before the present methods and devices are disclosed and described, it is to be understood that the methods and devices are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

In certain aspects of the present disclosure, implantable medical devices and surgical instruments having improved mechanical strength and biocompatibility are disclosed. In certain aspects of the present disclosure, the implantable medical devices and surgical instruments are not adversely affected by sterilization.

The medical device of some embodiments may be, without limitation, a surgical screw of any variety, a spinal or other orthopedic plate, a surgical rod, an interbody spinal device, a vertebral disc arthroplasty device, a nucleus replacement device, a corpectomy device, a vertebrectomy device, a mesh device, a facet fixation or arthroplasty device, a structural bone graft, a staple, a tether of synthetic material or wire, or other spinal fixation instrumentation, an intramedullary nail, an external fixation device, a hip prosthesis or therapeutic device, a knee prosthesis or therapeutic device, or an instrument useful with any of the previously recited devices.

The medical devices may also include neuromodulators including deep brain stimulators (DBS), various pain control devices, and lead systems for stimulation of the spinal cord, muscles, and other nerves of the body (such as, for instance, the vagal nerve); implantable diagnostic devices for monitoring cardiac function; cochlear implants; and drug pumps for administering periodic or demand based pharmacological therapy. Medical devices may also include gastric band systems, vascular access ports, injection ports, implantable cardioverter defibrillators, heart pacemaker, intra-uterine device, coronary stent, and tympanostomy tubes.

A wide variety of surgical instruments are contemplated for use by the present disclosure. Examples of surgical instruments for use in the present disclosure may include, but are not limited to various retractors, hemostats, tissue clamps, and needle holders. Surgical instruments may also include drills, reamers, implants, bone plates, scalpels, screws, etc. The term “surgical instrument” as used herein is intended to broadly mean any implement, workpiece or tool used during surgery either to shape, cut or form tissue or bone, or implanted or otherwise remain within tissue or bone.

In certain aspects, the surgical instruments may include any endoscopic surgical instruments including, but not limited to, laparoscopic or arthroscopic instruments. The surgical instrument may be any tool routinely used in endoscopic surgery, including, for example, tissue forceps, hemostats, retractors, clamps, scissors, needle holders and drivers, and cautery tools.

In certain aspects, the surgical instrument of the present disclosure may be formed from the polymer composition disclosed herein, either in whole or in part. In one aspect, the surgical instrument may include a handle and an operative end portion. In certain aspects, both the operative end portion and the handle may include polyetherimide. In certain aspects, only the operative end portion is composed of polyetherimide.

### Polymer Composition

In one aspect of the disclosure, the implantable medical device and surgical instrument may be formed using a polymer composition. In one aspect of the present disclosure, the polymer composition comprises a thermoplastic resin. Other components, however, may also be included in the thermoplastic resin. For example, the polymer composition may also include a ceramic and a metal. In one aspect of the disclosure, the polymer composition used to form the implantable medical device is MM (magnetic resonance imaging) compatible.

In one aspect of the disclosure, the polymer composition is suitable for melt processing such that the implantable medical device or surgical instrument may be formed using a melt process and in particular, injection molding.

The polymer composition may include any polymeric material known the art. The polymer composition may be composed of more than one polymeric material.

In one aspect of the disclosure, the polymers used in the polymer composition may be selected from a wide variety of thermoplastic polymers, and blends of thermoplastic polymers. The polymer composition can comprise a homopolymer, a copolymer such as a star block copolymer, a graft copolymer, an alternating block copolymer or a random copolymer, ionomer, dendrimer, or a combination comprising at least one of the foregoing. The polymer composition may also be a blend of polymers, copolymers, terpolymers, or the like, or a combination comprising at least one of the foregoing.

Examples of thermoplastic polymers that can be used in the polymer composition include polyacetals, polyacrylics, polycarbonates, polyalkyds, polystyrenes, polyolefins, polyesters, polyamides, polyaramides, polyamideimides, polyarylates, polyurethanes, epoxies, phenolics, silicones, polyarylsulfones, polyethersulfones, polyphenylene sulfides, polysulfones, polyimides, polyetherimides, polytetrafluoroethylenes, polyetherketones, polyether etherketones, polyether ketone ketones, polybenzoxazoles, polyoxadiazoles, polybenzothiazinophenothiazines, polybenzothiazoles, polypyrazinoquinoxalines, polypyromellitimides, polyquinoxalines, polybenzimidazoles, polyoxindoles, polyoxoisoindolines, polydioxoisoindolines, polytriazines, polypyridazines, polypiperazines, polypyridines, polypiperidines, polytriazoles, polypyrazoles, polycarboranes, polyoxabicyclononanes, polydibenzofurans, polyphthalides, polyacetals, polyanhydrides, polyvinyl ethers, polyvinyl thioethers, polyvinyl alcohols, polyvinyl ketones, polyvinyl halides, polyvinyl nitriles, polyvinyl esters, polysulfonates, polysulfides, poiythioesters, polysulfones, polysulfonamides, polyureas, polyphosphazenes, polysilazanes, polypropylenes, polyethylenes, polyethylene terephthalates, polyvinylidene fluorides, polysiloxanes, or the like, or a combination comprising at least one of the foregoing thermoplastic polymers.

Examples of blends of thermoplastic polymers that can be used polymer composition resin include acrylonitrile-butadiene-styrene/nylon, polycarbonate/acrylonitrile-butadiene-styrene, polyphenylene ether/polystyrene, polyphenylene ether/polyamide, polycarbonate/polyester, polyphenylene ether/polyolefin, or the like, or a combination comprising at least one of the foregoing.

In one aspect of the present disclosure, polymer composition may include, polycarbonates, polysulfones, polyesters, polyamides, polypropylene. In a further aspect, the polyimides used in the disclosed polymer composition may include polyamideimides, polyetherimides and polybenzimidazoles. In a further aspect, polyetherimides comprise melt processable polyetherimides.

### Polyetherimides

In one aspect of the disclosure, the polymer composition includes a polyetherimide. In an aspect, polyetherimides can comprise polyetherimides homopolymers (e.g., polyetherimidesulfones) and polyetherimides copolymers. The polyetherimide can be selected from (i) polyetherimidehomopolymers, e.g., polyetherimides, (ii) polyetherimide co-polymers, and (iii) combinations thereof. Polyetherimides are known polymers and are sold by SABIC Innovative Plastics under the ULTEM®*, EXTEM®*, and Siltem* brands (Trademark of SABIC Innovative Plastics IP B.V.).

In an aspect, the polyetherimides can be of formula (1):

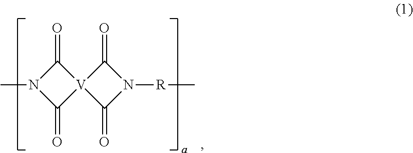

wherein a is more than 1, for example 10 to 1,000 or more, or more specifically 10 to 500.

The group V in formula (1) is a tetravalent linker containing an ether group (a “polyetherimide” as used herein) or a combination of an ether groups and arylenesulfone groups (a “polyetherimidesulfone”). Such linkers include but are not limited to: (a) substituted or unsubstituted, saturated, unsaturated or aromatic monocyclic and polycyclic groups having 5 to 50 carbon atoms, optionally substituted with ether groups, arylenesulfone groups, or a combination of ether groups and arylenesulfone groups; and (b) substituted or unsubstituted, linear or branched, saturated or unsaturated alkyl groups having 1 to 30 carbon atoms and optionally substituted with ether groups or a combination of ether groups, arylenesulfone groups, and arylenesulfone groups; or combinations comprising at least one of the foregoing. Suitable additional substitutions include, but are not limited to, ethers, amides, esters, and combinations comprising at least one of the foregoing.

The R group in formula (1) includes but is not limited to substituted or unsubstituted divalent organic groups such as: (a) aromatic hydrocarbon groups having 6 to 20 carbon atoms and halogenated derivatives thereof; (b) straight or branched chain alkylene groups having 2 to 20 carbon atoms; (c) cycloalkylene groups having 3 to 20 carbon atoms, or (d) divalent groups of formula (2):

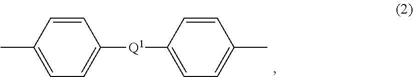

wherein Q1 includes but is not limited to a divalent moiety such as —O—, —S—, —C(O)—, —SO2-, —SO—, —CyH2y- (y being an integer from 1 to 5), and halogenated derivatives thereof, including perfluoroalkylene groups.

In an embodiment, linkers V include but are not limited to tetravalent aromatic groups of formula (3):

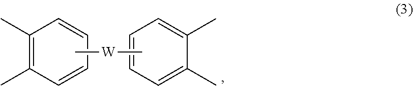

wherein W is a divalent moiety including —O—, —SO2-, or a group of the formula —O—Z—O— wherein the divalent bonds of the —O— or the —O—Z—O— group are in the 3,3′, 3,4′, 4,3′, or the 4,4′ positions, and wherein Z includes, but is not limited, to divalent groups of formulas (4):

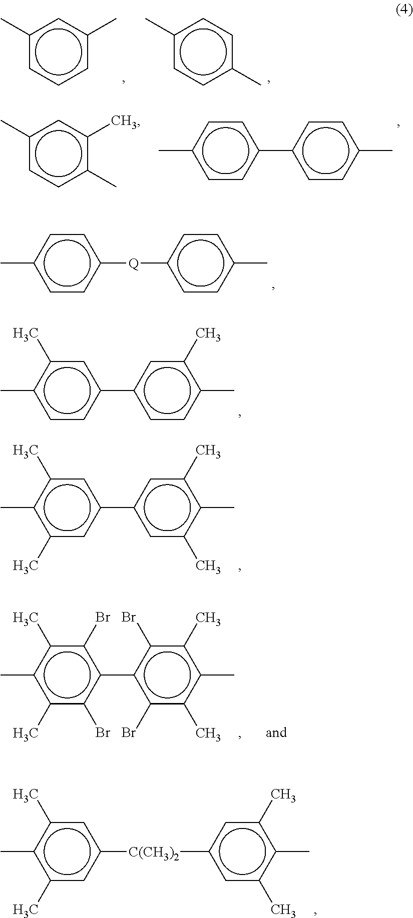

wherein Q includes, but is not limited to a divalent moiety including —O—, —S—, —C(O), —SO₂—, —SO—, —C_{y}H_{2y}— (y being an integer from 1 to 5), and halogenated derivatives thereof, including perfluoroalkylene groups.

In an aspect, the polyetherimide comprise more than 1, specifically 10 to 1,000, or more specifically, 10 to 500 structural units, of formula (5):

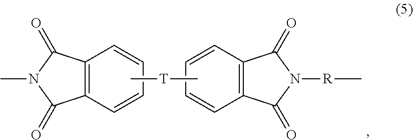

wherein T is —O— or a group of the formula —O—Z—O— wherein the divalent bonds of the —O— or the —O—Z—O— group are in the 3,3′, 3,4′, 4,3′, or the 4,4′ positions; Z is a divalent group of formula (3) as defined above; and R is a divalent group of formula (2) as defined above.

In another aspect, the polyetherimidesulfones are polyetherimides comprising ether groups and sulfone groups wherein at least 50 mole % of the linkers V and the groups R in formula (1) comprise a divalent arylenesulfone group. For example, all linkers V, but no groups R, can contain an arylenesulfone group; or all groups R but no linkers V can contain an arylenesulfone group; or an arylenesulfone can be present in some fraction of the linkers V and R groups, provided that the total mole fraction of V and R groups containing an aryl sulfone group is greater than or equal to 50 mole %.

Even more specifically, polyetherimidesulfones can comprise more than 1, specifically 10 to 1,000, or more specifically, 10 to 500 structural units of formula (6):

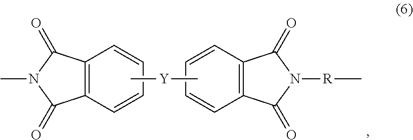

wherein Y is —O—, —SO2-, or a group of the formula —O—Z—O— wherein the divalent bonds of the —O—, SO2-, or the —O—Z—O— group are in the 3,3′, 3,4′, 4,3′, or the 4,4′ positions, wherein Z is a divalent group of formula (3) as defined above and R is a divalent group of formula (2) as defined above, provided that greater than 50 mole % of the sum of moles Y+moles R in formula (2) contain —SO2- groups.

It is to be understood that the polyetherimides and polyetherimidesulfones can optionally comprise linkers V that do not contain ether or ether and sulfone groups, for example linkers of formula (7):

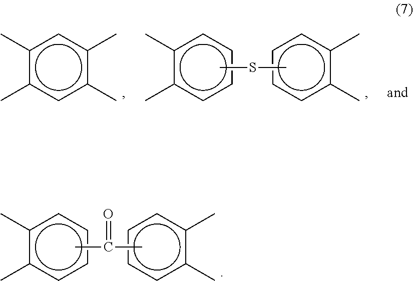

Imide units containing such linkers are generally be present in amounts ranging from 0 to 10 mole % of the total number of units, specifically 0 to 5 mole %. In one embodiment no additional linkers V are present in the polyetherimides and polyetherimidesulfones.

In another aspect, the polyetherimide comprises 10 to 500 structural units of formula (5) and the polyetherimidesulfone contains 10 to 500 structural units of formula (6).

Polyetherimides and polyetherimidesulfones can be prepared by any suitable process. In one embodiment, polyetherimides and polyetherimide copolymers include polycondensation polymerization processes and halo-displacement polymerization processes.

Polycondensation methods can include a method for the preparation of polyetherimides having structure (1) is referred to as the nitro-displacement process (X is nitro in formula (8)). In one example of the nitro-displacement process, N-methyl phthalimide is nitrated with 99% nitric acid to yield a mixture of N-methyl-4-nitrophthalimide (4-NPI) and N-methyl-3-nitrophthalimide (3-NPI). After purification, the mixture, containing approximately 95 parts of 4-NPI and 5 parts of 3-NPI, is reacted in toluene with the disodium salt of bisphenol-A (BPA) in the presence of a phase transfer catalyst. This reaction yields BPA-bisimide and NaNO2 in what is known as the nitro-displacement step. After purification, the BPA-bisimide is reacted with phthalic anhydride in an imide exchange reaction to afford BPA-dianhydride (BPADA), which in turn is reacted with a diamine such as meta-phenylene diamine (MPD) in ortho-dichlorobenzene in an imidization-polymerization step to afford the product polyetherimide.

Other diamines are also possible. Examples of suitable diamines include: m-phenylenediamine; p-phenylenediamine; 2,4-diaminotoluene; 2,6-diaminotoluene; m-xylylenediamine; p-xylylenediamine; benzidine; 3,3′-dimethylbenzidine; 3,3′-dimethoxybenzidine; 1,5-diaminonaphthalene; bis(4-aminophenyl)methane; bis(4-aminophenyl)propane; bis(4-aminophenyl)sulfide; bis(4-aminophenyl)sulfone; bis(4-aminophenyl)ether; 4,4′-diaminodiphenylpropane; 4,4′-diaminodiphenylmethane(4,4′-methylenedianiline); 4,4′-diaminodiphenylsulfide; 4,4′-diaminodiphenylsulfone; 4,4′-diaminodiphenylether(4,4′-oxydianiline); 1,5-diaminonaphthalene; 3,3′dimethylbenzidine; 3-methylheptamethylenediamine; 4,4-dimethylheptamethylenediamine; 2,2′,3,3′-tetrahydro-3,3,3′,3′-tetramethyl-1,1′-spirobi[1H-indene]-6,6′-diamine; 3,3′,4,4′-tetrahydro-4,4,4′,4′-tetramethyl-2,2′-spirobi[2H-1-benzo-pyran]-7,7′-diamine; 1,1′-bis[1-amino-2-methyl-4-phenyl]cyclohexane, and isomers thereof as well as mixtures and blends comprising at least one of the foregoing. In one embodiment, the diamines are specifically aromatic diamines, especially m- and p-phenylenediamine and mixtures comprising at least one of the foregoing.

Suitable dianhydrides that can be used with the diamines include and are not limited to 2,2-bis[4-(3,4-dicarboxyphenoxy)phenyl]propane dianhydride; 4,4′-bis(3,4-dicarboxyphenoxy)diphenyletherdianhydride; 4,4′-bis(3,4-dicarboxyphenoxy)diphenylsulfidedianhydride; 4,4′-bis(3,4-dicarboxyphenoxy)benzophenonedianhydride; 4,4′-bis(3,4-dicarboxyphenoxy)diphenylsulfonedianhydride; 2,2-bis[4-(2,3-dicarboxyphenoxy)phenyl]propane dianhydride; 4,4′-bis(2,3-dicarboxyphenoxy)diphenyletherdianhydride; 4,4′-bis(2,3-dicarboxyphenoxy)diphenylsulfidedianhydride; 4,4′-bis(2,3-dicarboxyphenoxy)benzophenonedianhydride; 4,4′-bis(2,3-dicarboxyphenoxy)diphenylsulfonedianhydride; 4-(2,3-dicarboxyphenoxy)-4′-(3,4-dicarboxyphenoxy)diphenyl-2,2-propane dianhydride; 4-(2,3-dicarboxyphenoxy)-4′-(3,4-dicarboxyphenoxy)diphenyletherdianhydride; 4-(2,3-dicarboxyphenoxy)-4′-(3,4-dicarboxyphenoxy)diphenylsulfide dianhydride; 4-(2,3-dicarboxyphenoxy)-4′-(3,4-dicarboxyphenoxy)benzophenonedianhydride; 4-(2,3-dicarboxyphenoxy)-4′-(3,4-dicarboxyphenoxy)diphenylsulfone dianhydride; 1,3-bis(2,3-dicarboxyphenoxy)benzene dianhydride; 1,4-bis(2,3-dicarboxyphenoxy)benzene dianhydride; 1,3-bis(3,4-dicarboxyphenoxy)benzene dianhydride; 1,4-bis(3,4-dicarboxyphenoxy)benzene dianhydride; 3,3′,4,4′-diphenyl tetracarboxylicdianhydride; 3,3′,4,4′-benzophenonetetracarboxylic dianhydride; naphthalicdianhydrides, such as 2,3,6,7-naphthalic dianhydride, etc.; 3,3′,4,4′-biphenylsulphonictetracarboxylic dianhydride; 3,3′,4,4′-biphenylethertetracarboxylic dianhydride; 3,3′,4,4′-dimethyldiphenylsilanetetracarboxylic dianhydride; 4,4′-bis (3,4-dicarboxyphenoxy)diphenylsulfidedianhydride; 4,4′-bis (3,4-dicarboxyphenoxy)diphenyl sulphonedianhydride; 4,4′-bis (3,4-dicarboxyphenoxy)diphenylpropanedianhydride; 3,3′,4,4′-biphenyltetracarboxylic dianhydride; bis(phthalic)phenylsulphineoxidedianhydride; p-phenylene-bis(triphenylphthalic)dianhydride; m-phenylene-bis(triphenylphthalic)dianhydride; bis(triphenylphthalic)-4,4′-diphenylether dianhydride; bis(triphenylphthalic)-4,4′-diphenylmethane dianhydride; 2,2′-bis(3,4-dicarboxyphenyl)hexafluoropropanedianhydride; 4,4′-oxydiphthalic dianhydride; pyromelliticdianhydride; 3,3′,4,4′-diphenylsulfonetetracarboxylic dianhydride; 4′,4′-bisphenol A dianhydride; hydroquinone diphthalic dianhydride; 6,6′-bis(3,4-dicarboxyphenoxy)-2,2′,3,3′-tetrahydro-3,3,3′,3′-tetramethyl-1,1′-spirobi[1H-indene]dianhydride; 7,7′-bis(3,4-dicarboxyphenoxy)-3,3′,4,4′-tetrahydro-4,4,4′,4′-tetramethyl-2,2′-spirobi[2H-1-benzopyran]dianhydride; 1,1′-bis[1-(3,4-dicarboxyphenoxy)-2-methyl-4-phenyl]cyclohexane dianhydride; 3,3′,4,4′-diphenylsulfonetetracarboxylic dianhydride; 3,3′,4,4′-diphenylsulfidetetracarboxylic dianhydride; 3,3′,4,4′-diphenylsulfoxidetetracarboxylic dianhydride; 4,4′-oxydiphthalic dianhydride; 3,4′-oxydiphthalic dianhydride; 3,3′-oxydiphthalic dianhydride; 3,3′-benzophenonetetracarboxylic dianhydride; 4,4′-carbonyldiphthalic dianhydride; 3,3′,4,4′-diphenylmethanetetracarboxylic dianhydride; 2,2-bis(4-(3,3-dicarboxyphenyl)propane dianhydride; 2,2-bis(4-(3,3-dicarboxyphenyl)hexafluoropropanedianhydride; (3,3′,4,4′-diphenyl)phenylphosphinetetracarboxylicdianhydride; (3,3′,4,4′-diphenyl)phenylphosphineoxidetetracarboxylicdianhydride; 2,2′-dichloro-3,3′,4,4′-biphenyltetracarboxylic dianhydride; 2,2′-dimethyl-3,3′,4,4′-biphenyltetracarboxylic dianhydride; 2,2′-dicyano-3,3′,4,4′-biphenyltetracarboxylic dianhydride; 2,2′-dibromo-3,3′,4,4′-biphenyltetracarboxylic dianhydride; 2,2′-diiodo-3,3′,4,4′-biphenyltetracarboxylic dianhydride; 2,2′-ditrifluoromethyl-3,3′,4,4′-biphenyltetracarboxylic dianhydride; 2,2′-bis(1-methyl-4-phenyl)-3,3′,4,4′-biphenyltetracarboxylic dianhydride; 2,2′-bis(1-trifluoromethyl-2-phenyl)-3,3′,4,4′-biphenyltetracarboxylic dianhydride; 2,2′-bis(1-trifluoromethyl-3-phenyl)-3,3′,4,4′-biphenyltetracarboxylic dianhydride; 2,2′-bis(1-trifluoromethyl-4-phenyl)-3,3′,4,4′-biphenyltetracarboxylic dianhydride; 2,2′-bis(1-phenyl-4-phenyl)-3,3′,4,4′-biphenyltetracarboxylic dianhydride; 4,4′-bisphenol A dianhydride; 3,4′-bisphenol A dianhydride; 3,3′-bisphenol A dianhydride; 3,3′,4,4′-diphenylsulfoxidetetracarboxylic dianhydride; 4,4′-carbonyldiphthalic dianhydride; 3,3′,4,4′-diphenylmethanetetracarboxylic dianhydride; 2,2′-bis(1,3-trifluoromethyl-4-phenyl)-3,3′,4,4′-biphenyltetracarboxylic dianhydride, and all isomers thereof, as well as combinations of the foregoing.

Halo-displacement polymerization methods for making polyetherimides and polyetherimidesulfones include and are not limited to, the reaction of a bis(phthalimide) for formula (8):

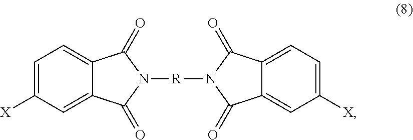

wherein R is as described above and X is a nitro group or a halogen. Bis-phthalimides (8) can be formed, for example, by the condensation of the corresponding anhydride of formula (9):

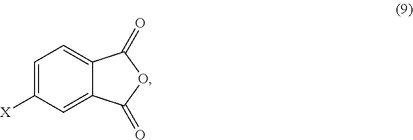

wherein X is a nitro group or halogen, with an organic diamine of the formula (10):

H₂N—R—NH₂ (10),
wherein R is as described above.

Illustrative examples of amine compounds of formula (10) include: ethylenediamine, propylenediamine, trimethylenediamine, diethylenetriamine, triethylenetetramine, hexamethylenediamine, heptamethylenediamine, octamethylenediamine, nonamethylenediamine, decamethylenediamine, 1,12-dodecanediamine, 1,18-octadecanediamine, 3-methylheptamethylenediamine, 4,4-dimethylheptamethylenediamine, 4-methylnonamethylenediamine, 5-methylnonamethylenediamine, 2,5-dimethylhexamethylenediamine, 2,5-dimethylheptamethylenediamine, 2, 2-dimethylpropylenediamine, N-methyl-bis (3-aminopropyl) amine, 3-methoxyhexamethylenediamine, 1,2-bis(3-aminopropoxy) ethane, bis(3-aminopropyl) sulfide, 1,4-cyclohexanediamine, bis-(4-aminocyclohexyl) methane, m-phenylenediamine, p-phenylenediamine, 2,4-diaminotoluene, 2,6-diaminotoluene, m-xylylenediamine, p-xylylenediamine, 2-methyl-4,6-diethyl-1,3-phenylene-diamine, 5-methyl-4,6-diethyl-1,3-phenylene-diamine, benzidine, 3,3′-dimethylbenzidine, 3,3′-dimethoxybenzidine, 1,5-diaminonaphthalene, bis(4-aminophenyl) methane, bis(2-chloro-4-amino-3, 5-diethylphenyl) methane, bis(4-aminophenyl) propane, 2,4-bis(b-amino-t-butyl) toluene, bis(p-b-amino-t-butylphenyl) ether, bis(p-b-methyl-o-aminophenyl) benzene, bis(p-b-methyl-o-aminopentyl) benzene, 1, 3-diamino-4-isopropylbenzene, bis(4-aminophenyl) ether and 1,3-bis(3-aminopropyl) tetramethyldisiloxane. Mixtures of these amines can be used. Illustrative examples of amine compounds of formula (10) containing sulfone groups include but are not limited to, diaminodiphenylsulfone (DDS) and bis(aminophenoxy phenyl) sulfones (BAPS). Combinations comprising any of the foregoing amines can be used.

The polyetherimides can be synthesized by the reaction of the bis(phthalimide) (8) with an alkali metal salt of a dihydroxy substituted aromatic hydrocarbon of the formula HO—V—OH wherein V is as described above, in the presence or absence of phase transfer catalyst. Suitable phase transfer catalysts are disclosed in U.S. Pat. No. 5,229,482. Specifically, the dihydroxy substituted aromatic hydrocarbon a bisphenol such as bisphenol A, or a combination of an alkali metal salt of a bisphenol and an alkali metal salt of another dihydroxy substituted aromatic hydrocarbon can be used.

In one embodiment, the polyetherimide comprises structural units of formula (5) wherein each R is independently p-phenylene or m-phenylene or a mixture comprising at least one of the foregoing; and T is group of the formula —O—Z—O— wherein the divalent bonds of the —O—Z—O— group are in the 3,3′ positions, and Z is 2,2-diphenylenepropane group (a bisphenol A group). Further, the polyetherimidesulfone comprises structural units of formula (6) wherein at least 50 mole % of the R groups are of formula (4) wherein Q is —SO2- and the remaining R groups are independently p-phenylene or m-phenylene or a combination comprising at least one of the foregoing; and T is group of the formula —O—Z—O— wherein the divalent bonds of the —O—Z—O— group are in the 3,3′ positions, and Z is a 2,2-diphenylenepropane group.

The polyetherimide and polyetherimidesulfone can be used alone or in combination with each other and/or other of the disclosed polymeric materials in fabricating the polymeric components of the invention. In one embodiment, only the polyetherimide is used. In another embodiment, the weight ratio of polyetherimide: polyetherimidesulfone can be from 99:1 to 50:50.

The polyetherimides can have a weight average molecular weight (Mw) of 5,000 to 100,000 grams per mole (g/mole) as measured by gel permeation chromatography (GPC). In some embodiments the Mw can be 10,000 to 80,000. The molecular weights as used herein refer to the absolute weight averaged molecular weight (Mw).

The polyetherimides can have an intrinsic viscosity greater than or equal to 0.2 deciliters per gram (dl/g) as measured in m-cresol at 25° C. Within this range the intrinsic viscosity can be 0.35 to 1.0 dl/g, as measured in m-cresol at 25° C.

The polyetherimides can have a glass transition temperature of greater than 180° C., specifically of 200° C. to 500° C., as measured using differential scanning calorimetry (DSC) per ASTM test D3418. In some embodiments, the polyetherimide and, in particular, a polyetherimide has a glass transition temperature of 240 to 350° C.

The polyetherimides can have a melt index of 0.1 to 10 grams per minute (g/min), as measured by American Society for Testing Materials (ASTM) DI 238 at 340 to 370° C., using a 6.7 kilogram (kg) weight.

In certain aspects, the polyetherimides of the present disclosure may be unfilled, standard flow grades (PEI-1 in Tables 1-2) or unfilled, high flow grades (PEI-2 in Tables 1-2), or may be filled, for example, with carbon (e.g., carbon fiber) or glass. Filled polymer components may include between 40 wt % and 90 wt % of the polyetherimide resin and between 10 wt % and 60 wt % of a filler by weight of the polymer component. Other formulations may be used.

An alternative halo-displacement polymerization process for making polyetherimides, e.g., polyetherimides having structure (1) is a process referred to as the chloro-displacement process (X is Cl in formula (8)). The chloro-displacement process is illustrated as follows: 4-chloro phthalic anhydride and meta-phenylene diamine are reacted in the presence of a catalytic amount of sodium phenyl phosphinate catalyst to produce the bischlorophthalimide of meta-phenylene diamine (CAS No. 148935-94-8). The bischlorophthalimide is then subjected to polymerization by chloro-displacement reaction with the disodium salt of BPA in the presence of a catalyst in ortho-dichlorobenzene or anisole solvent. Alternatively, mixtures of 3-chloro- and 4-chlorophthalic anhydride may be employed to provide a mixture of isomeric bischlorophthalimides which may be polymerized by chloro-displacement with BPA disodium salt as described above.

Siloxane polyetherimides can include polysiloxane/polyetherimide block or random copolymers having a siloxane content of greater than 0 and less than 40 weight percent (wt %) based on the total weight of the block copolymer. The block copolymer comprises a siloxane block of Formula (I):

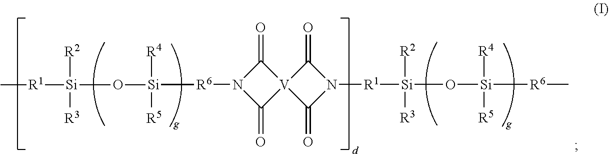

wherein R¹⁻⁶ are independently at each occurrence selected from the group consisting of substituted or unsubstituted, saturated, unsaturated, or aromatic monocyclic groups having 5 to 30 carbon atoms, substituted or unsubstituted, saturated, unsaturated, or aromatic polycyclic groups having 5 to 30 carbon atoms, substituted or unsubstituted alkyl groups having 1 to 30 carbon atoms and substituted or unsubstituted alkenyl groups having 2 to 30 carbon atoms, V is a tetravalent linker selected from the group consisting of substituted or unsubstituted, saturated, unsaturated, or aromatic monocyclic and polycyclic groups having 5 to 50 carbon atoms, substituted or unsubstituted alkyl groups having 1 to 30 carbon atoms, substituted or unsubstituted alkenyl groups having 2 to 30 carbon atoms and combinations comprising at least one of the foregoing linkers, g equals 1 to 30, and d is 2 to 20. Commercially available siloxane polyetherimides can be obtained from SABIC Innovative Plastics under the brand name SILTEM* (*Trademark of SABIC Innovative Plastics IP B.V.)

The polyetherimide resin can have a weight average molecular weight (Mw) within a range having a lower limit and/or an upper limit. The range can include or exclude the lower limit and/or the upper limit. The lower limit and/or upper limit can be selected from 5000, 6000, 7000, 8000, 9000, 10000, 11000, 12000, 13000, 14000, 15000, 16000, 17000, 18000, 19000, 20000, 21000, 22000, 23000, 24000, 25000, 26000, 27000, 28000, 29000, 30000, 31000, 32000, 33000, 34000, 35000, 36000, 37000, 38000, 39000, 40000, 41000, 42000, 43000, 44000, 45000, 46000, 47000, 48000, 49000, 50000, 51000, 52000, 53000, 54000, 55000, 56000, 57000, 58000, 59000, 60000, 61000, 62000, 63000, 64000, 65000, 66000, 67000, 68000, 69000, 70000, 71000, 72000, 73000, 74000, 75000, 76000, 77000, 78000, 79000, 80000, 81000, 82000, 83000, 84000, 85000, 86000, 87000, 88000, 89000, 90000, 91000, 92000, 93000, 94000, 95000, 96000, 97000, 98000, 99000, 100000, 101000, 102000, 103000, 104000, 105000, 106000, 107000, 108000, 109000, and 110000 daltons. For example, the polyetherimide resin can have a weight average molecular weight (Mw) from 5,000 to 100,000 daltons, from 5,000 to 80,000 daltons, or from 5,000 to 70,000 daltons. The primary alkyl amine modified polyetherimide will have lower molecular weight and higher melt flow than the starting, unmodified, polyetherimide.

The polyetherimide resin can be selected from the group consisting of a polyetherimide, for example as described in U.S. Pat. Nos. 3,875,116; 6,919,422 and 6,355,723 a silicone polyetherimide, for example as described in U.S. Pat. Nos. 4,690,997; 4,808,686 a polyetherimidesulfone resin, as described in U.S. Pat. No. 7,041,773 and combinations thereof, each of these patents are incorporated herein their entirety.

The polyetherimide resin can have a glass transition temperature within a range having a lower limit and/or an upper limit. The range can include or exclude the lower limit and/or the upper limit. The lower limit and/or upper limit can be selected from 100, 110, 120, 130, 140, 150, 160, 170, 180, 190, 200, 210, 220, 230, 240, 250, 260, 270, 280, 290, 300 and 310 degrees Celsius. For example, the polyetherimide resin can have a glass transition temperature (Tg) greater than about 200 degrees Celsius.

The polyetherimide resin can be substantially free (less than 100 ppm) of benzylic protons. The polyetherimide resin can be free of benzylic protons. The polyetherimide resin can have an amount of benzylic protons below 100 ppm. In one embodiment, the amount of benzylic protons ranges from more than 0 to below 100 ppm. In another embodiment, the amount of benzylic protons is not detectable.

The polyetherimide resin can be substantially free (less than 100 ppm) of halogen atoms. The polyetherimide resin can be free of halogen atoms. The polyetherimide resin can have an amount of halogen atoms below 100 ppm. In one embodiment, the amount of halogen atoms range from more than 0 to below 100 ppm. In another embodiment, the amount of halogen atoms is not detectable.

### Therapeutic Agents

In certain aspects of the disclosure, the implantable medical device may additionally include certain therapeutic agents. For example, therapeutic agents that are commonly used to promote bone fusion or ingrowth may be used. Such therapeutic agents may include natural or synthetic therapeutic agents such as hone morphogenic proteins (BMPs), growth factors, bone marrow aspirate, stem cells, progenitor cells, antibiotics, or other osteoconductive, osteoinductive, osteogenic, or any other fusion enhancing material or beneficial therapeutic agent.

In one aspect, the implantable medical device includes a coating formed on surfaces of the implantable medical device. The coating, for example, may be a biomimetic and/or osteogenic (e.g., bone morphogenetic protein(s) (BMP) and related compounds) coating. In certain aspects, the coating may be used to enhance bone growth on the implantable medical device. In some aspects, the coating may be formed on substantially all of the surfaces of the implantable medical device; though, in other aspects, only a portion of the surfaces are coated; and, in some embodiments, the implantable medical device may not be coated at all. Suitable coating materials include calcium phosphate, BMP and related compounds, amongst others.

In some aspects, a substance (e.g., a drug) may elute from the implantable medical device and/or a coating on the implantable medical device. For example, a substance incorporated into the implantable medical device and/or coating may be emitted into regions around the implantable medical device. In some aspects, the substance (e.g., BMP and related compounds) may be selected to enhance bone growth. The substance, for example, may be incorporated at different concentrations into different locations of the implantable medical device and/or coating.

In certain aspects of the disclosure, the polymer composition may also include a biocide. The biocide may be selected from germicides, antimicrobials, antibiotics, antibacterials, antiyeasts, antialgals, antivirals, antifungals, antiprotozoals, antiparasites, and combinations thereof.

In certain aspects of the disclosure, the implantable medical device or surgical instrument may be formed by any method or combination of methods known in the art. These methods include, but are not limited to, molding processes, additive manufacturing, and machining. These molding processes include, but are not limited to, various melt forming process, injection molding, blow molding (stretch, extrusion or injection), sheet and film extrusion, profile extrusion, thermoforming, additive manufacturing, compression molding, fiber extrusion, powder sintering, transfer molding, reaction injection (RIM) molding, vacuum forming, cold casting, dip molding, slush molding and press molding. In one aspect, a combination of these molding methods may be used to form the implantable medical device or surgical instrument.

Various surgical instruments are contemplated by the present disclosure. For example, a screw driver, a distractor, a reamer, a ring curette, a holder, a graft pusher, an impactor, a forked impactor, and/or a final impactor may be used.

In certain aspects of the disclosure, the surgical instruments may also be formed using the polymer composition disclosed herein. The implantable medical device of this or any other aspect of the disclosure may be any implant or instrument used to accomplish a medical procedure. The medical device of some aspects of the disclosure is capable of undergoing one or more sterilizations, without degrading in a manner that would make the device unsuitable for use in a medical procedure. The sterilizations may be from steam autoclave sterilization cycles or from application of a chemical sterilizing substance, or from any other effective sterilization substance or process, including, dry heat, ethylene oxide gas, vaporized hydrogen peroxide, or other sterilization procedures.

### Aspects

The present disclosure comprises at least the following aspects.

Aspect 1. An implantable medical device formed from a polymer composition comprising a polyetherimide.

Aspect 2. An implantable medical device formed from a polymer composition comprising a polyetherimide having structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 3. An implantable medical device formed from a polymer composition comprising a polyetherimide having a weight average molecular weight of at least about 10,000 to about 150.00 grams per mole (g/mol).

Aspect 4. The implantable medical device of any preceding aspect, wherein the implantable medical device comprises a surgical screw, an orthopedic plate, a surgical rod, a vertebral disc arthroplasty device, a nucleus replacement device, a corpectomy device, a vertebrectomy device, a mesh device, a facet fixation device, an arthroplasty device, a structural bone graft, a staple, a tether of synthetic material, an intramedullary nail, an external fixation device, a hip prosthesis, or a knee prosthesis.

Aspect 5. The implantable medical device of any one of aspects 1-3, wherein the implantable medical device comprises a deep brain stimulators (DBS), an implantable diagnostic devices for monitoring cardiac function, a cochlear implant, or a drug pump.

Aspect 6. The implantable medical device of any preceding aspect, wherein the polyetherimide has less than 100 ppm amine end groups.

Aspect 7. The implantable medical device of any preceding aspect, further comprising a biocide disposed on a surface of the implantable medical device, wherein the biocide is selected from germicides, antimicrobials, antibiotics, antibacterials, antiyeasts, antialgals, antivirals, antifungals, antiprotozoals, antiparasites, and combinations thereof.

Aspect 8. The implantable medical device of any preceding aspect, wherein the implantable medical device is formed from a polymer component comprising between 40 wt % and 90 wt % of the polyetherimide and between 10 wt % and 60 wt % of a filler by weight of the polymer component.

Aspect 9. The method of aspect 8, wherein the filler comprises glass, carbon, carbon fiber, or a combination thereof.

Aspect 10. The implantable medical device of any preceding aspect, wherein the polymer composition further comprises ceramic or metal.

Aspect 11. The implantable medical device of any preceding aspect, wherein polyetherimide comprises repeating units of the formula

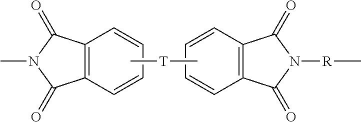

wherein R is a divalent radical of the formula

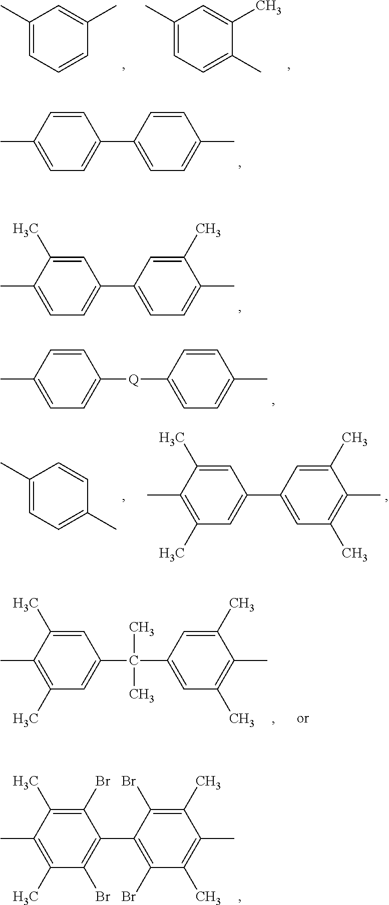

or combinations thereof wherein Q is selected from —O—, —S—, —C(O)—, —SO₂—, —SO—, and —C_{y}H_{2y}— wherein y is an integer from 1 to 5; and T is —O— or a group of the formula —O—Z—O— wherein the divalent bonds of the —O— or the —O—Z—O— group are in the 3,3′, 3,4′, 4,3′, or the 4,4′ positions and Z is a divalent group of the formula

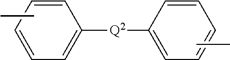

wherein Q² is selected from —O—, —S—, —C(O)—, —SO₂—, —SO—, and —C_{y}H_{2y}— wherein y is an integer from 1 to 5.

Aspect 12. The implantable medical device according to any of the preceding aspects, wherein the device is sterilized using at least one sterilization process selected from the group consisting of: steam autoclave sterilization, hydrogen peroxide sterilization, gamma-ray sterilization and ethylene oxide sterilization.

Aspect 13. The implantable medical device according to any of the preceding aspects, wherein the medical device has a compressive strength after sterilization that is within 5% of the compressive strength of the medical device prior to sterilization.

Aspect 14. A surgical instrument formed from a polymer composition comprising a polyetherimide.

Aspect 15. A surgical instrument formed from a polymer composition comprising a polyetherimide having structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

Aspect 16. The surgical instrument of any one of aspects 14-15, wherein the polyetherimide has a weight average molecular weight of at least about 10,000 to about 150.00 grams per mole (g/mol).

Aspect 17. The surgical instrument of any one of aspects 14-16, wherein the surgical instrument is an endoscopic surgical instrument.

Aspect 18. The surgical instrument of any one of aspects 14-16, wherein the surgical instrument is a retractor, hemostat, tissue clamp, or needle holder.

Aspect 19. The surgical instrument of any one of aspects 14-18, further comprising a biocide disposed on a surface of the surgical instrument, wherein the biocide is selected from germicides, antimicrobials, antibiotics, antibacterials, antiyeasts, antialgals, antivirals, antifungals, antiprotozoals, antiparasites, and combinations thereof.

Aspect 20. The surgical instrument of any one of aspects 14-19, wherein the surgical instrument is formed from a polymer component comprising between 40 wt % and 90 wt % of the polyetherimide and between 10 wt % and 60 wt % of a filler by weight of the polymer component.

Aspect 21. The surgical instrument of aspect 20, wherein the filler comprises glass, carbon, carbon fiber, or a combination thereof.

Aspect 22. The surgical instrument of any one of aspects 14-21, wherein polyetherimide comprises repeating units of the formula

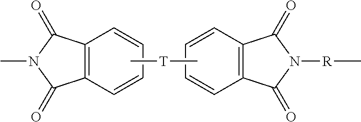

wherein R is a divalent radical of the formula

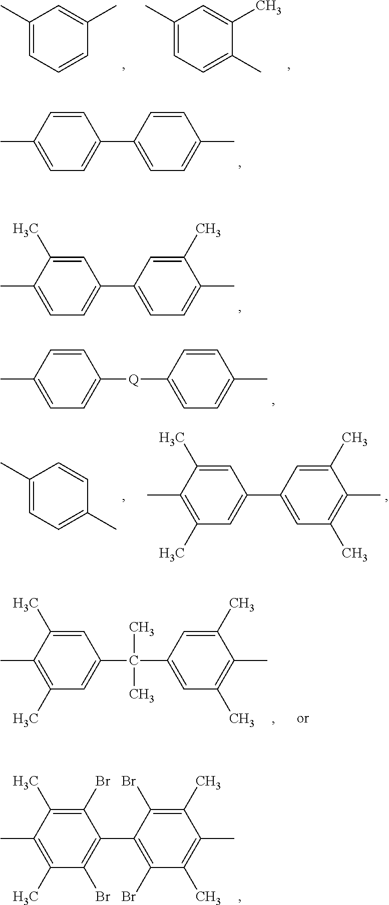

or combinations thereof wherein Q is selected from —O—, —S—, —C(O)—, —SO₂—, —SO—, and —C_{y}H_{2y}— wherein y is an integer from 1 to 5; and T is —O— or a group of the formula —O—Z—O— wherein the divalent bonds of the —O— or the —O—Z—O— group are in the 3,3′, 3,4′, 4,3′, or the 4,4′ positions and Z is a divalent group of the formula

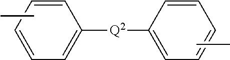

wherein Q² is selected from —O—, —S—, —C(O)—, —SO₂—, —SO—, and —C_{y}H_{2y}— wherein y is an integer from 1 to 5.

As an illustrative example, the polyetherimides used in forming the apparatus of the present disclosure may exhibit distinguishable properties over other comparative polymers, as shown in Tables 1-2 (PEI—polyetherimide; PPSU—polyphenylsulfone; PSU—polysulfone; PEEK—Polyether ether ketone; TPU—thermoplastic polyurethane):

TABLE 1

| | | | E1 | E2 | CE1 | CE2 | CE3 |
|---|---|---|---|---|---|---|---|
| | | | Polymer Type | | | | |
| MECHANICAL | Unit | Standard | PEI-1 | PEI-2 | PPSU | PSU | PEEK |
| Tensile Stress @ | kgf/cm² | ASTM D | 1120 | 1120 | 710 | 720 | 1020 |
| Yield, Type I, 5 | | 638 | | | | | |
| mm/min | | | | | | | |
| Tensile Modulus, | kgf/cm² | ASTM D | 36500 | 36500 | 23900 | 25300 | 37700 |
| 5 mm/min | | 638 | | | | | |
| Flexural Stress | kgf/cm² | ASTM D | 1760 | 1770 | 930 | 1080 | 1560 |
| @ Yield, 1.3 | | 790 | | | | | |
| mm/min, 50 mm | | | | | | | |
| span | | | | | | | |
| Flexural | kgf/cm² | ASTM D | 35000 | 34900 | 24600 | 27400 | 38700 |
| Modulus, 1.3 | | 790 | | | | | |
| mm/min, 50 mm | | | | | | | |
| span | | | | | | | |

| IMPACT | Unit | Standard | Value | | | | |
|---|---|---|---|---|---|---|---|
| Izod Impact, | cm- | ASTM D | 5 | 3 | 70 | 7.0 | 5.4 |
| notched, 23° C. | kgf/cm | 256 | | | | | |

| PHYSICAL | Unit | Standard | | Value | | | |
|---|---|---|---|---|---|---|---|
| Specific Gravity | — | ASTM D | 1.27 | 1.27 | 1.29 | 1.24 | 1.30 |
| | | 792 | | | | | |
| Melt Flow Rate, | g/10 min | ASTM D | — | — | — | — | 36 |
| 400° C./2.16 kgf | | 1238 | | | | | |
| Melt Flow Rate, | g/10 min | ASTM D | — | — | 14-20 | — | — |
| 365° C./5.0 kgf | | 1238 | | | | | |
| Melt Flow Rate, | g/10 min | ASTM D | — | — | — | 6.5 | — |
| 343° C./2.16 kgf | | 1238 | | | | | |
| Melt Flow Rate, | g/10 min | ASTM D | 9 | 17.8 | — | — | — |
| 337° C./6.6 kgf | | 1238 | | | | | |

| ELECTRICAL | Unit | Standard | | Value | | | |
|---|---|---|---|---|---|---|---|
| Volume | Ohm- | ASTM D | 1.00E+17 | 1.00E+17 | 9.00E+15 | 3.00E+16 | — |
| Resistivity | cm | 257 | | | | | |

| THERMAL | Unit | Standard | | Value | | | |
|---|---|---|---|---|---|---|---|
| Glass Transition | ° C. | | 217 | 217 | 220 | — | 147 |
| Temperature | | | | | | | |
| Heat Deflection | ° C. | ASTM D | 201 | 198 | 207 | 174 | 160 |
| Temperature, | | 648 | | | | | |
| 1.82 MPa | | | | | | | |

TABLE 2

| | | | E1 | E2 | CE4 | CE5 | CE6 |
|---|---|---|---|---|---|---|---|
| | | | Polymer Type | | | | |
| MECHANICAL | Unit | Standard | PEI-1 | PEI-2 | TPU | TPU | TPU |
| Tensile Stress @ | kgf/cm² | ASTM D | — | — | — | 720 | 1020 |
| Yield, Type I, 5 | | 638 | | | | | |
| mm/min | | | | | | | |
| Tensile Modulus, | kgf/cm² | ASTM D | — | — | — | 25300 | 37700 |
| 5 mm/min | | 638 | | | | | |
| Flexural Stress | kgf/cm² | ASTM D | 16 | 63 | 770 | 1080 | 1560 |
| @ Yield, 1.3 | | 790 | | | | | |
| mm/min, 50 mm | | | | | | | |
| span | | | | | | | |
| Flexural | kgf/cm² | ASTM D | 370 | 1520 | 20320 | 27400 | 38700 |
| Modulus, 1.3 | | 790 | | | | | |
| mm/min, 50 mm | | | | | | | |
| span | | | | | | | |

| IMPACT | Unit | Standard | | | | | |
|---|---|---|---|---|---|---|---|
| Izod Impact, | cm- | ASTM D | — | — | — | 7.0 | 5.4 |
| notched, 23° C. | kgf/cm | 256 | | | | | |

| PHYSICAL | Unit | Standard | | | | | |
|---|---|---|---|---|---|---|---|
| Specific Gravity | — | ASTM D | 1.12 | 1.16 | 1.19 | 1.24 | 1.30 |
| | | 792 | | | | | |
| Melt Flow Rate, | g/10 min | ASTM D | — | — | — | — | — |
| 400° C./2.16 kgf | | 1238 | | | | | |
| Melt Flow Rate, | g/10 min | ASTM D | — | — | — | — | — |
| 365° C./5.0 kgf | | 1238 | | | | | |
| Melt Flow Rate, | g/10 min | ASTM D | — | — | — | — | — |
| 343° C./2.16 kgf | | 1238 | | | | | |
| Melt Flow Rate, | g/10 min | ASTM D | 9 | 17.8 | — | — | — |
| 337° C./6.6 kgf | | 1238 | | | | | |
| Melt Flow Rate, | g/10 min | ASTM D | — | — | 17 | 13 | 37 |
| 224° C. | | 1238 | | | | | |

| ELECTRICAL | Unit | Standard | | | | | |
|---|---|---|---|---|---|---|---|
| Volume | Ohm- | ASTM D | — | — | — | 3.00E+16 | — |
| Resistivity | cm | 257 | | | | | |

| THERMAL | Unit | Standard | | | | | |
|---|---|---|---|---|---|---|---|
| Glass Transition | ° C. | — | — | — | — | — | 147 |
| Temperature | | | | | | | |
| Heat Deflection | ° C. | ASTM D | — | — | — | 174 | 160 |
| Temperature, | | 648 | | | | | |
| 1.82 MPa | | | | | | | |

It will be appreciated that the foregoing description provides examples of the disclosed system and technique. However, it is contemplated that other implementations of the disclosure may differ in detail from the foregoing examples. All references to the disclosure or examples thereof are intended to reference the particular example being discussed at that point and are not intended to imply any limitation as to the scope of the disclosure more generally. All language of distinction and disparagement with respect to certain features is intended to indicate a lack of preference for those features, but not to exclude such from the scope of the disclosure entirely unless otherwise indicated.

1. An implantable medical device formed from a polymer composition comprising a polyetherimide having structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

2. An implantable medical device formed from a polymer composition comprising a polyetherimide having a weight average molecular weight of at least about 10,000 to about 150.00 grams per mole (g/mol).

3. The implantable medical device of claim 1, wherein the implantable medical device comprises a surgical screw, an orthopedic plate, a surgical rod, a vertebral disc arthroplasty device, a nucleus replacement device, a corpectomy device, a vertebrectomy device, a mesh device, a facet fixation device, an arthroplasty device, a structural bone graft, a staple, a tether of synthetic material, an intramedullary nail, an external fixation device, a hip prosthesis, or a knee prosthesis.

4. The implantable medical device of claim 1, wherein the implantable medical device comprises a deep brain stimulators (DBS), an implantable diagnostic devices for monitoring cardiac function, a cochlear implant, or a drug pump.

5. The implantable medical device of claim 1, wherein the polyetherimide has less than 100 ppm amine end groups.

6. The implantable medical device of claim 1, further comprising a biocide disposed on a surface of the implantable medical device, wherein the biocide is selected from germicides, antimicrobials, antibiotics, antibacterials, antiyeasts, antialgals, antivirals, antifungals, antiprotozoals, antiparasites, and combinations thereof.

7. The implantable medical device of claim 1, wherein the implantable medical device is formed from a polymer component comprising between 40 wt % and 90 wt % of the polyetherimide and between 10 wt % and 60 wt % of a filler by weight of the polymer component.

8. The method of claim 7, wherein the filler comprises glass, carbon, carbon fiber, or a combination thereof.

9. The implantable medical device of claim 1, wherein the polymer composition further comprises ceramic or metal.

10. The implantable medical device of claim 1, wherein polyetherimide comprises repeating units of the formula 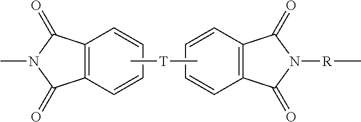
wherein R is a divalent radical of the formula 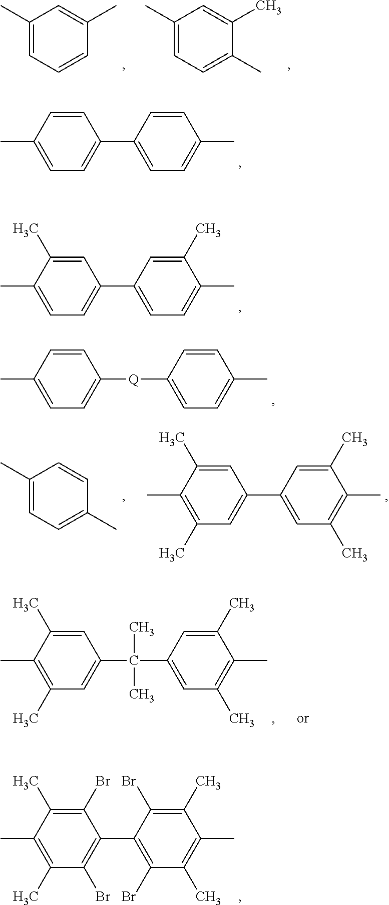
or combinations thereof wherein Q is selected from —O—, —S—, —C(O)—, —SO₂—, —SO—, and —C_{y}H_{2y}— wherein y is an integer from 1 to 5; and T is —O— or a group of the formula —O—Z—O— wherein the divalent bonds of the —O— or the —O—Z—O— group are in the 3,3′, 3,4′, 4,3′, or the 4,4′ positions and Z is a divalent group of the formula 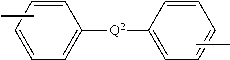
wherein Q² is selected from —O—, —S—, —C(O)—, —SO₂—, —SO—, and —C_{y}H_{2y}— wherein y is an integer from 1 to 5.

11. The implantable medical device claim 1, wherein the device is sterilized using at least one sterilization process selected from the group consisting of: steam autoclave sterilization, hydrogen peroxide sterilization, gamma-ray sterilization and ethylene oxide sterilization.

12. The implantable medical device claim 1, wherein the medical device has a compressive strength after sterilization that is within 5% of the compressive strength of the medical device prior to sterilization.

13. A surgical instrument formed from a polymer composition comprising a polyetherimide having structural units derived from at least one diamine selected from 1,3-diaminobenzene, 1,4-diaminobenzene, 4,4′-diaminodiphenyl sulfone, oxydianiline, 1,3-bis(4-aminophenoxy)benzene, or combinations thereof.

14. The surgical instrument of claim 13, wherein the polyetherimide has a weight average molecular weight of at least about 10,000 to about 150.00 grams per mole (g/mol).

15. The surgical instrument of claim 13, wherein the surgical instrument is an endoscopic surgical instrument.

16. The surgical instrument of claim 13, wherein the surgical instrument is a retractor, hemostat, tissue clamp, or needle holder.

17. The surgical instrument of claim 13, further comprising a biocide disposed on a surface of the surgical instrument, wherein the biocide is selected from germicides, antimicrobials, antibiotics, antibacterials, antiyeasts, antialgals, antivirals, antifungals, antiprotozoals, antiparasites, and combinations thereof.

18. The surgical instrument of claim 13, wherein the surgical instrument is formed from a polymer component comprising between 40 wt % and 90 wt % of the polyetherimide and between 10 wt % and 60 wt % of a filler by weight of the polymer component.

19. The surgical instrument of claim 18, wherein the filler comprises glass, carbon, carbon fiber, or a combination thereof.

20. The surgical instrument of claim 13, wherein polyetherimide comprises repeating units of the formula 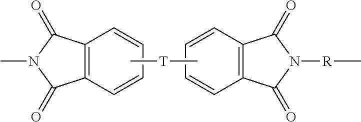
wherein R is a divalent radical of the formula 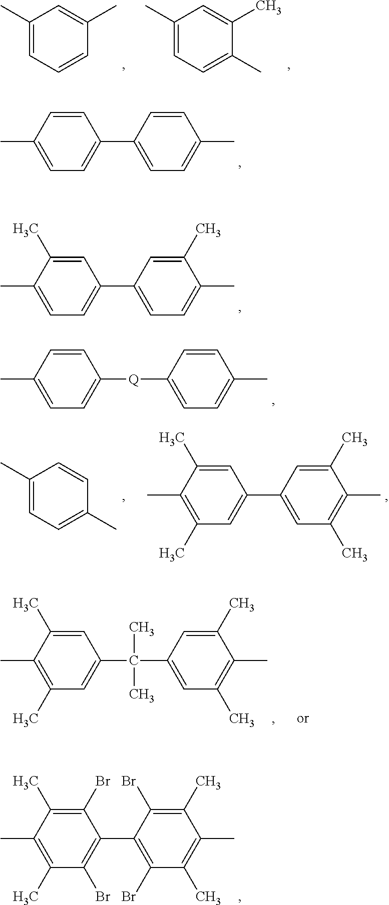
or combinations thereof wherein Q is selected from —O—, —S—, —C(O)—, —SO₂—, —SO—, and —C_{y}H_{2y}— wherein y is an integer from 1 to 5; and T is —O— or a group of the formula —O—Z—O— wherein the divalent bonds of the —O— or the —O—Z—O— group are in the 3,3′, 3,4′, 4,3′, or the 4,4′ positions and Z is a divalent group of the formula 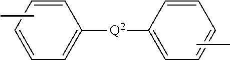
wherein Q² is selected from —O—, —S—, —C(O)—, —SO₂—, —SO—, and —C_{y}H_{2y}— wherein y is an integer from 1 to 5.
